(19) United States
(12) Patent Application Publication
(10) Pub. No.: US 2026/0208173 A1
(43) Pub. Date: Jul. 23, 2026

(54) ALKOXYLATION PROCESSES USING PHOSPHORUS AND DOUBLE METAL CYANIDE CATALYSTS

(71) Applicant: DOW GLOBAL TECHNOLOGIES LLC, Midland, MI (US)

(72) Inventors: David K. STEELMAN, Freeport, TX (US); Alex J. KOSANOVICH, Freeport, TX (US); Arjun RAGHURAMAN, Lake Jackson, TX (US); Peter J. WALLER, Midland, MI (US)

(21) Appl. No.: 19/140,987

(22) PCT Filed: Dec. 11, 2023
(86) PCT No.: PCT/US2023/083369

§ 371 Date: Jun. 18, 2025

Related U.S. Application Data
(60) Provisional application No. 63/433,548, filed on Dec. 19, 2022.

(51) Int. Cl. B01J 31/22; B01J 27/26; B01J 31/02; C07C 41/03; C08G 65/26

(52) U.S. Cl. CPC B01J 31/2208; B01J 27/26; B01J 31/0268; C07C 41/03; C08G 65/2663

(57) ABSTRACT

Starter compounds are alkoxylated in a multi-step process. A first alkoxylation step is performed in the presence of certain phosphorus catalysts, to produce an intermediate having a molecular weight of 400 to 1500 g/mol. A second alkoxylation step is performed in the presence of a double metal cyanide catalyst. Residues of the phosphorus catalyst(s) can be left in the intermediate and be present during the second alkoxylation step, so expensive finishing steps can be avoided between the two alkoxylation steps. Similarly, residues of both the phosphorus and double metal cyanide catalysts can be left in the final product.

This invention relates to an alkoxylation process in which a cyclic oxide is added onto a starter compound to produce an ether or polyether.

Polyethers are produced globally in large quantities. Polyether polyols, for example, are an important raw material for producing polyurethanes. Among other things, they are used to make high resiliency, molded, or rigid foams. Polyether monols are used, for example, as surfactants and industrial solvents, among other uses. Carbonate- and ester-modified alkylene oxide polymers also find uses in these and other applications.

Polyether monols and polyols are produced via alkoxylation of a starter compound, in which an active site on the starter reacts with a cyclic oxide in a ring-opening reaction. A terminal hydroxyl group is produced, which in turn can function as an active site for a subsequent alkoxylation step, thereby producing a polyether chain. The active site of the starter compound is a group containing an active hydrogen, such as a hydroxyl or thiol group. The main functions of the starter compound are to provide molecular weight control and to establish the number of hydroxyl groups the alkoxylated product will have.

A catalyst is needed to obtain economical polymerization rates. The most commonly used catalysts are alkali metal hydroxides such as potassium hydroxide and the so-called double metal cyanide (DMC) catalyst complexes, of which zinc hexacyanocobaltate catalyst complexes are the most commercially important type.

Alkali metal hydroxides provide the benefits of low catalyst costs and acceptable alkoxylation rates. They are versatile in that they effectively polymerize many alkylene oxides. Nonetheless, alkali metal hydroxides have well-known drawbacks. The alkoxylated product must be neutralized, and catalyst residues scrupulously removed. These finishing steps add greatly to both capital and operating costs and produce additional waste streams that must be cleaned up and/or disposed of.

DMC catalysts provide rapid polymerization rates compared to alkali metal catalysts, even when used at very low catalyst concentrations. An important advantage of DMC catalysts over alkali metal hydroxides is no neutralization step is needed. The catalyst residues often can be left in the product, unlike the case when alkali metal hydroxides are used as the polymerization catalyst. This can result in significantly lower production costs. Nonetheless, the DMC catalysts have significant disadvantages as well. They tend to perform poorly in the presence of high concentrations of hydroxyl groups, and especially in the presence of low molecular weight starter compounds like glycerin or sorbitol that have hydroxyl groups in the 1,2- or 1,3-positions with respect to each other. Under these conditions, the catalysts are difficult to activate, perform sluggishly and often deactivate before the polymerization is completed. This represents a significant limitation on the widespread adoption of DMC catalysts. It is often necessary to produce the polyether in two or more discrete steps, in which the early stages of the polymerization are conducted in the presence of an alkali metal catalyst and, after cleaning up the resulting intermediate product, the remainder of the polymerization is performed using the DMC catalyst. This approach requires the intermediate to be neutralized and purified (because the DMC catalyst is deactivated by strong bases), thus re-introducing costs which the DMC-catalyzed polymerization is intended to avoid.

Certain Lewis acids have been evaluated as alkylene oxide polymerization catalysts. The Lewis acids require essentially no activation time, but deactivate rapidly and therefore cannot produce high molecular weight polymers or high conversions of alkylene oxide to polymer. Another problem with many Lewis acid catalysts is that they deactivate at higher operating temperatures. This disqualifies them for use with certain starters that are solids, viscous, or otherwise poorly miscible with the cyclic oxide, because in those cases high operating temperatures are needed to melt the starter, reduce its viscosity or promote mixing with the cyclic oxide.

Various phosphonium compounds have been described in the literature. See, for example, Science 341 1374 (2013), Dalton Trans. 2018, 47, 11411, Chem. Eur. J. 2015, 21, 6491-6500, Dalton Trans. 2016, 45, 5568, Angew. Chem. Int. Ed. 2014, 53, 6538-6541, Chem. Sci. 2015, 6, 2016 and Chem. Commun., 2018, 54, 662-665. They have been described for use as catalysts in various reactions such as olefin isomerization, hydrosilylation, dehydrocoupling, hydrodefluorination, hydrogenation and Friedel-Crafts reactions. Angew. Chem. Int. Ed. 2014, 53, 6538-6541 describes the use of a phosphonium catalyst to polymerize tetrahydrofuran in the absence of starter to produce an 86,000 molecular weight polymer with high polydispersity.

This invention is an alkoxylation process, comprising (step I) forming a reaction mixture comprising a) a starter compound having at least one hydroxyl or thiol group and a molecular weight of up to 300; b) at least one cyclic oxide and c) a catalytically effective amount of a phosphorus catalyst;
(step II) reacting the cyclic oxide b) with the starter compound in the presence of the phosphorus catalyst to form an intermediate alkoxylated product having a number average molecular weight of 400 to 1500 containing residues of the phosphorus catalyst;
(step III) further alkoxylating the intermediate alkoxylated product containing residues of the phosphorus catalyst in the presence of a catalytically effective amount of a double metal cyanide catalyst complex to produce a polyether having a number average molecular weight at least 200 g/mol greater than that of the intermediate alkoxylated product,
wherein the phosphorus catalyst has any of structures

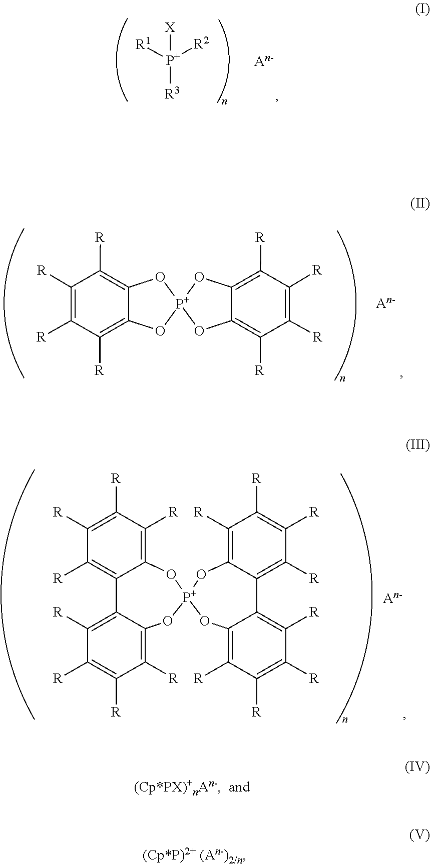

wherein R¹, R² and R³ independently are groups having unsubstituted or substituted, optionally heteroatomic, aromatic five- or six-member rings having a direct bond between a carbon atom of the optionally heteroatomic aromatic five- or six-member ring and the phosphorus atom, each R is independently hydrogen, halogen, unsubstituted or inertly substituted C₁₋₁₂ alkoxyl, aryloxy, unsubstituted or inertly substituted linear, branched and/or cyclic alkyl and unsubstituted or inertly substituted aryl, with the proviso that any two R groups may together form a ring structure, Cp* designates an optionally substituted cyclopentadienyl ligand, X is halogen, hydroxyl, unsubstituted or inertly substituted alkyl, unsubstituted or inertly substituted aryl, unsubstituted or inertly substituted alkoxy, or unsubstituted or inertly substituted aryloxy, A represents a weakly coordinating anion, n− represents the valence of A and n represents the absolute value of the valence of A.

The process of the invention overcomes major deficiencies of prior art processes. The sluggish polymerization rates and need for catalyst removal inherent in alkali metal polymerizations are avoided. The poor performance of double metal cyanide catalysts in alkoxylating very low molecular weight starters, especially those having hydroxyl groups in the 1, 2- and 1,3-positions, is avoided. Surprisingly, residues of the phosphorus catalyst used in step (II) of the process do not interfere with the performance of the double metal cyanide catalyst, and therefore do not need to be removed from the intermediate alkoxylated product before step (III) is performed, or afterward. The polyether produced in Step III does not require treatment to remove catalyst residues before being used in many applications, particularly the manufacture of polyurethanes.

Another surprising result of the invention is that the proportion of primary hydroxyl groups is unexpectedly high when 1,2-propylene oxide is homopolymerized in step (III) of the process. In conventional processes, about 8 to 10% of the hydroxyl groups formed are primary when 1,2-propylene oxide is polymerized using a double metal cyanide catalyst. With this invention, the proportion of primary hydroxyl groups formed is significantly higher, often being 15 to 25% or even more. The increased proportion of primary hydroxyl groups increases the reactivity of the polyether product, which is often quite desirable when producing polyurethanes by reaction of the polyether with a polyisocyanate. Significantly, this increase in primary hydroxyl content and reactivity is achieved without polymerizing ethylene oxide in step (III) of the process, and without performing a subsequent ethylene oxide capping step.

In some embodiments, the phosphorus catalyst has structure I, wherein R¹, R² and R³ independently are groups having unsubstituted or substituted, optionally heteroatomic, aromatic five- or six-member rings having a direct bond between a carbon atom of the optionally heteroatomic aromatic five- or six-member ring and the phosphorus atom. Inert substituents do not react with the starter or cyclic oxide under the conditions of the alkoxylation reaction and include, for example, alkyl (linear, branched and/or cyclic), aryl, ether (—O—), ester (—O—C(O)—), carbonate (—O—C(O)—O))—, halogen (especially F, Cl, Br and/or I), sulfide (—S—), polysulfide (—S_{z}—, where z>1), amino, silyl and the like. R¹, R² and R³ preferably do not contain active sites such as —OH, —NH, —SH or —COOH where alkoxylation can take place, and preferably do not contain cyclic oxide structures.

In some embodiments, the phosphorus catalyst has structure I, wherein R¹, R² and R³ each are independently selected from the group consisting of phenyl and phenyl substituted with one or more substituents selected from the group consisting of halogen, unsubstituted or inertly substituted C₁₋₁₂ alkyl, unsubstituted or inertly substituted C₁₋₁₂ alkoxyl, or trifluoromethyl groups. If a C₁₋₁₂ alkyl or C₁₋₁₂ alkoxyl group has more than 2 carbon atoms, it may be linear, branched and/or cyclic. A C₁₋₁₂ alkyl or C₁₋₁₂ alkoxyl group may be substituted with inert substituents as described above, particularly halogen and especially F, Cl or Br. A substituted phenyl group, for example may be substituted in the para-position (relative to the bond to the central phosphorus atom) with an unsubstituted or inertly substituted C₁₋₁₂ alkoxyl group and in such a case optionally contains no other substituents. In specific embodiments, R¹, R² and R³ are independently selected from phenyl, pentafluorophenyl, 3,5-bis(trifluoromethyl)phenyl or 4-alkoxyphenyl wherein the alkoxy group has 1 to 4 carbon atoms, preferably 1 or 2 carbon atoms.

In other embodiments, the phosphorus catalyst has structure I, wherein R¹, R² and R³ are unsubstituted or substituted, optionally heteroatomic, aromatic five-member rings having a direct bond between a carbon atom of the optionally heteroatomic aromatic five-member ring and the phosphorus atom. R¹, R² and R³ may be the same or different and each may be, for example:

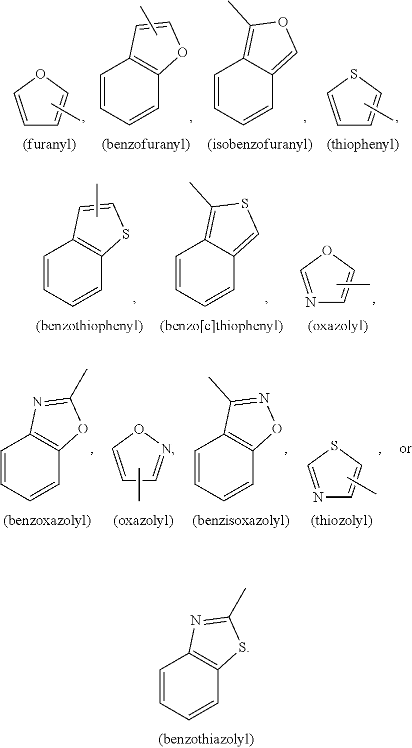

In any of the foregoing, any ring carbon can be unsubstituted or substituted with an inert substituent.

In other embodiments, the phosphorus catalyst has structure I, wherein at least one of R¹, R² and R³ is a unsubstituted or substituted, optionally heteroatomic, aromatic five-member ring having a direct bond between a carbon atom of the optionally heteroatomic aromatic five-member ring and the phosphorus atom, such as those described above, and at least one of R¹, R² and R³ is a unsubstituted or substituted, optionally heteroatomic, aromatic six-member ring having a direct bond between a carbon atom of the optionally heteroatomic aromatic six-member ring and the phosphorus atom.

X in Structure I is halogen, hydroxyl, unsubstituted or inertly substituted alkyl, unsubstituted or inertly substituted alkoxy, unsubstituted or inertly substituted aryl or unsubstituted or inertly substituted aryloxy. X is preferably F, Cl, Br, I, OCH₃, OC₂H₅, phenoxy or CF₃.

In other embodiments, the phosphorus catalyst has either of Structures II or III. Examples of R groups in Structures II and III include hydrogen, F, Cl, Br and I. Other examples of R groups include —O—R² wherein R² is unsubstituted or inertly substituted linear, branched and/or cyclic alkyl having 1 to 12 carbon atoms (especially 1-4 carbon atoms such as methyl, ethyl, n-propyl, isopropyl, n-butyl, sec-butyl and t-butyl). Any two R groups, particularly any two adjacent R groups, may together form a ring, it being understood that such ring structure includes carbon atoms of the benzene ring to which the R groups are bonded. Such cyclic structures may be aliphatic or aromatic. If R² is substituted, a preferred substituent is halogen, especially F or Cl. Specific examples of halogen-substituted R² groups include —CF₃, —CCl₃, perfluoroethyl, perchloroethyl, monochloromethyl, monofluoromethyl and the like.

In still other embodiments, the phosphorus catalyst has Structure IV or V. The Cp* ligand in Structure IV and V has the structure

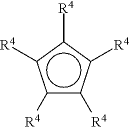

wherein each R⁴ is independently hydrogen or an inert substituent. Inert substituents do not react with the starter or cyclic oxide under the conditions of the alkoxylation reaction and include, for example, alkyl (linear, branched and/or cyclic), aryl, alkyl-substituted aryl, aryl-substituted alkyl, halogen (especially F, Cl, Br), alkoxyl, oxyaryl, and the like. For example, the R³4 groups may be selected from hydrogen, F, Cl, C₁₋₄ alkyl, phenyl, methoxy or ethoxy. In some embodiments, all R⁴ groups are the same. Any two R⁴ groups, particularly any two adjacent R⁴ groups, may together form a cyclic structure at includes the carbon atoms of the cyclopentadienyl ring to which the R⁴ groups are bonded. Such cyclic structures may be aliphatic or aromatic. In specific embodiments, Cp is cyclopentadienyl, pentafluorocyclopentadienyl, pentachlorocyclopentadienyl, pentamethylcyclopentadienyl or pentaphenylcyclopentadienyl.

(Cp*PX)⁺ₙ cations are monocationic and may exist in an η²-Cp* configuration. (Cp*P)²⁺ cations are dicationic and may exist in an η²-Cp configuration. Dicationic phosphorus catalysts corresponding to the structure (Cp*P)²⁺(Aⁿ⁻)_{2/n} may in addition have one or more solvent ligands coordinated to the structure.

X in Structures IV and V is as described with regard to Structure I.

The anion A is a weakly coordinating anion that has a valence of n⁻. n in structures I, II and III is the absolute value of the valence of the anion A. n− is preferably 1⁻ or 2⁻ and most preferably 1⁻. Weakly coordinating anions are those whose coordination to the associated cation is weaker than that of the surrounding solvent molecules. Coordination strength of an anion is conveniently determined by forming a tri-n-octylammonium salt of the anion, dissolving the salt in carbon tetrachloride, and measuring the N—H stretching frequency by infrared spectroscopy, using a method as described, for example, in J. Am. Chem Soc. 2006, 128, 8500-8508. An N—H stretching frequency of 3000 cm⁻¹ or greater, especially 3050 cm⁻¹ or greater, is indicative of a weakly coordinating anion.

Examples of weakly coordinating anions include tetrakis(perfluorophenyl)borate, tetrakis[3,5-bis(trifluoromethyl)phenyl]borate, trifluoromethanesulfonate (triflate), Al[OC(CF₃)₃]₄—, HCB₁₁Me₅F₆⁻, B₁₂F₁₂²⁻, HCB₁₁H₅F₆, B(OTeF₅)₄—, B(OTeF₅)₆—, Sb(OTeF₅)₄—, Sb(OTeF₅)₆—, Al[OC(CF₃)₃]₄—, Al[OCH(CF₃)₂]₄— and Al[OC(CH₃)(CF₃)₂]₄—.

Specific examples of phosphorus catalysts include:

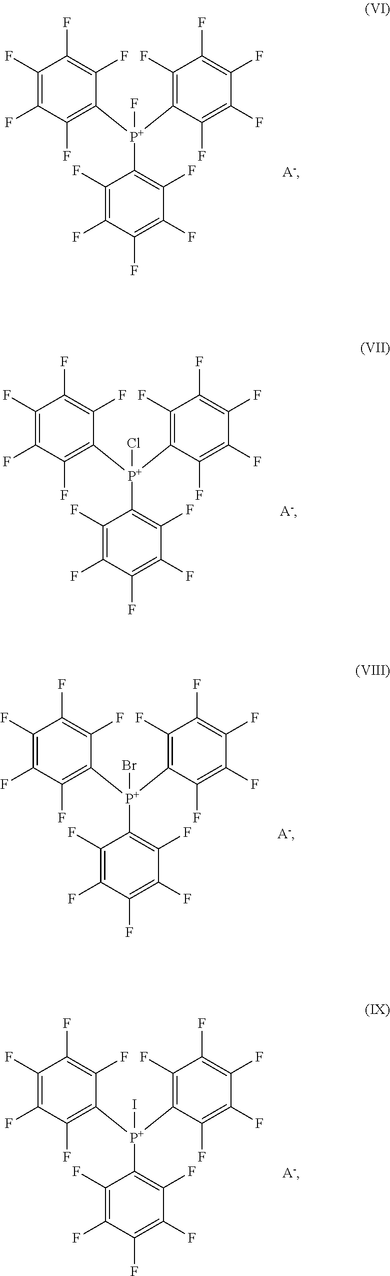

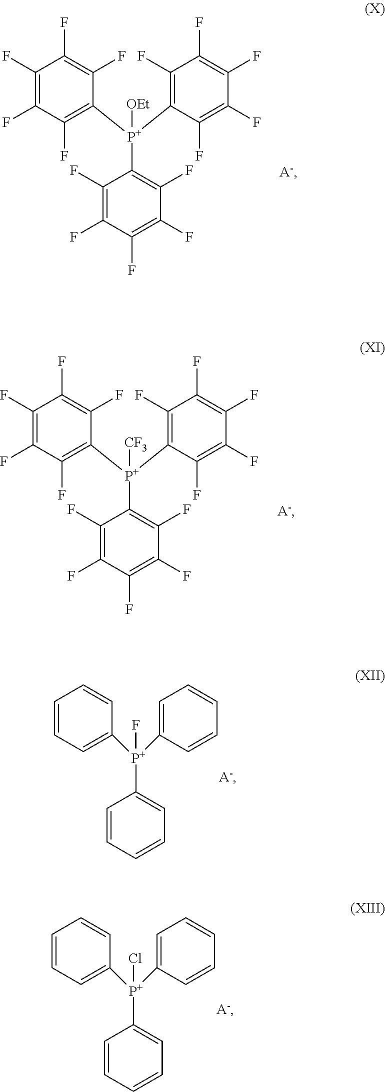

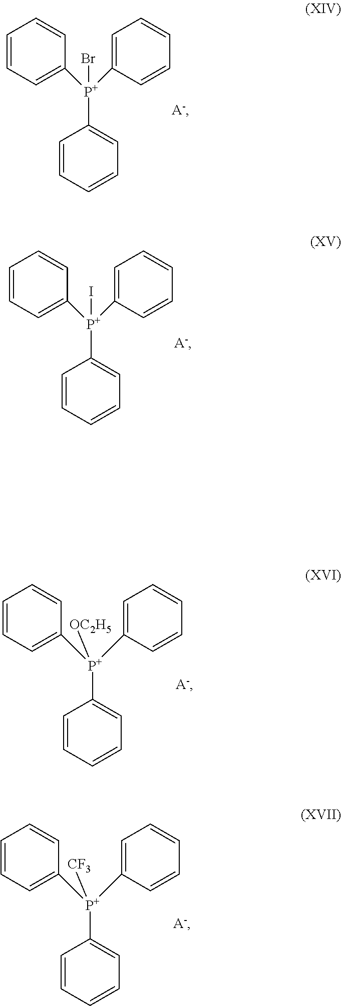

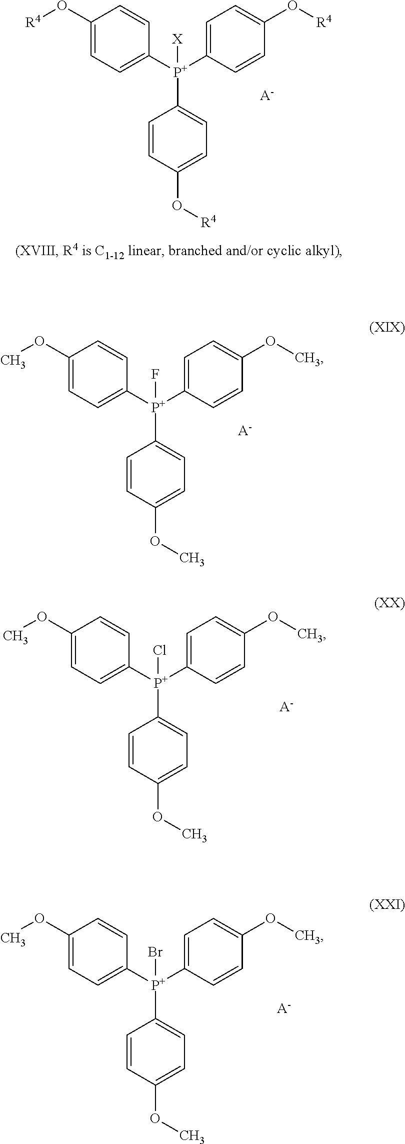

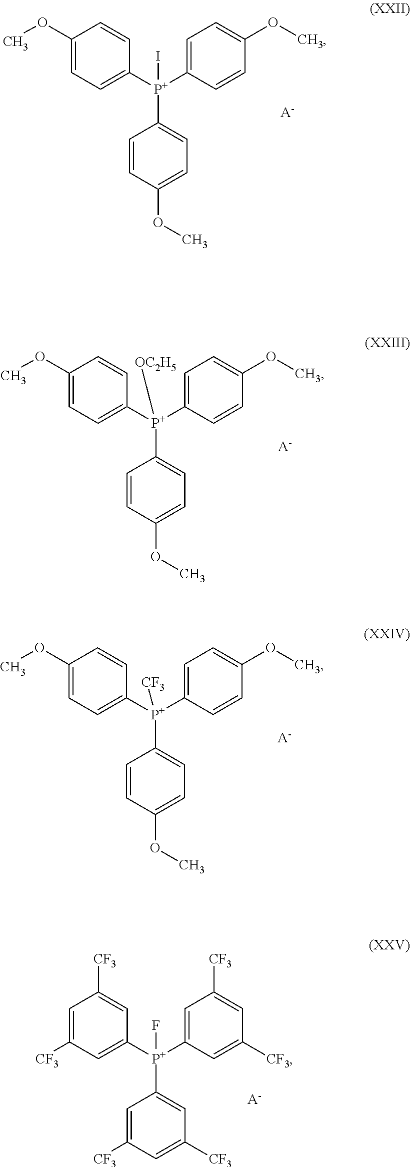

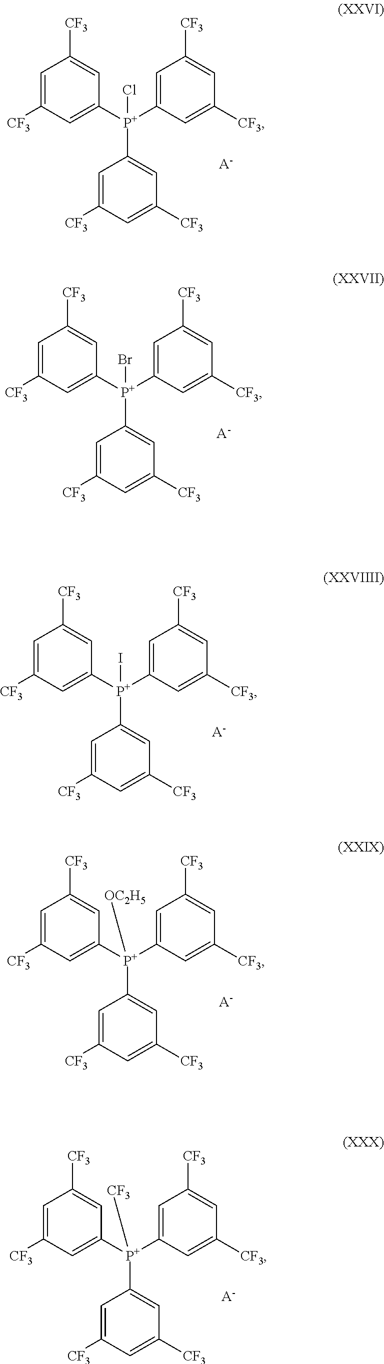

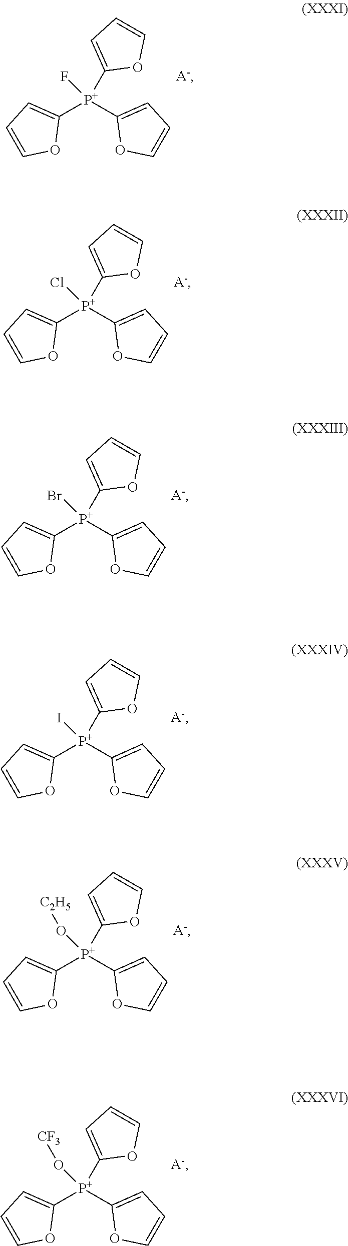

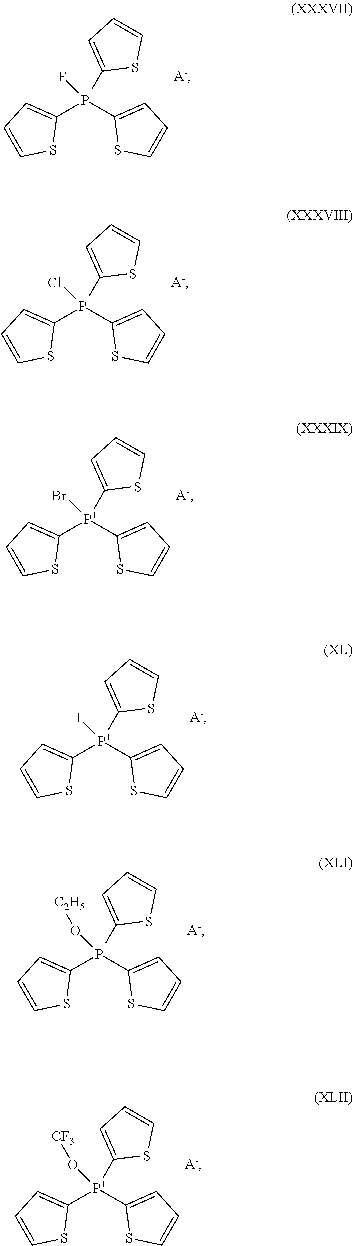

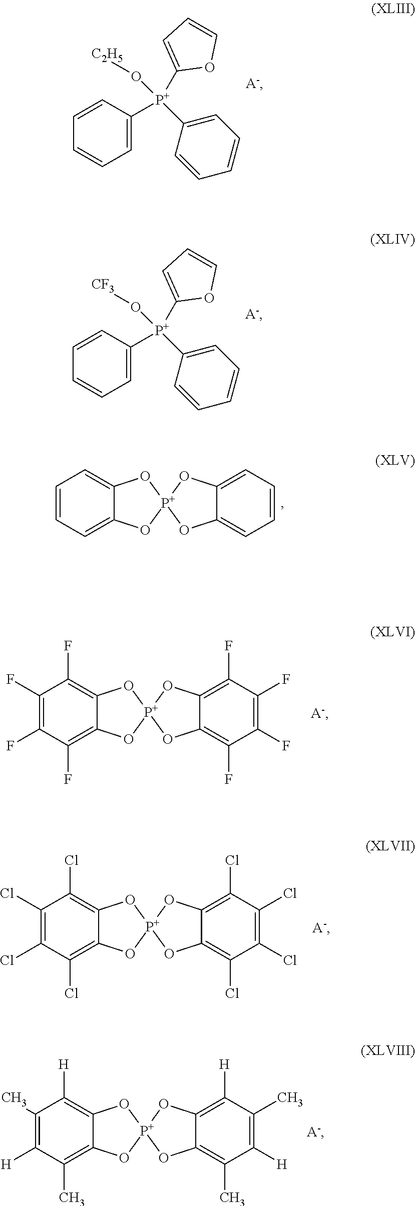

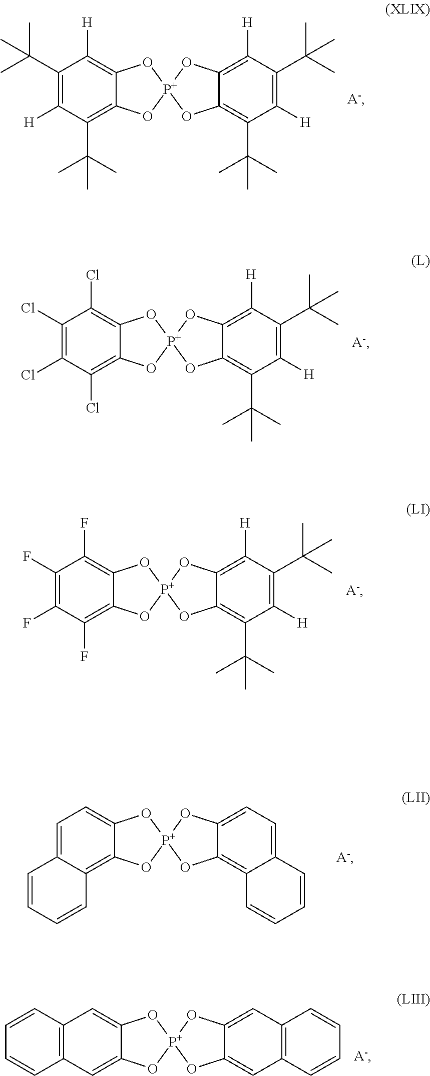

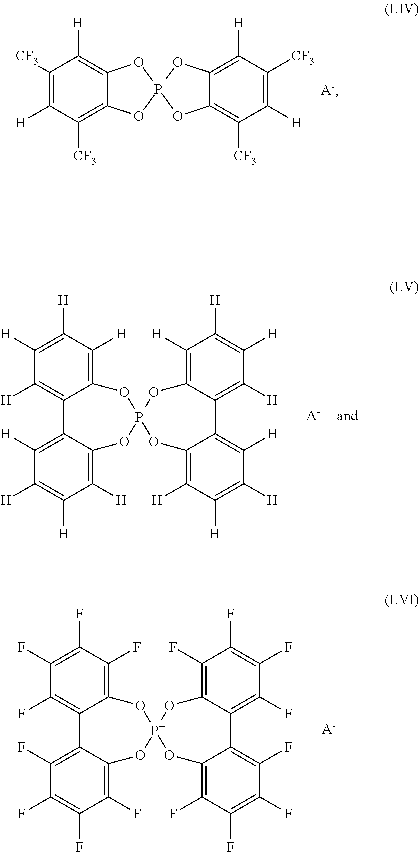

and the like, wherein each case A is a monovalent anion. Analogous compounds of the form Z⁺₂A²⁻, where Z⁺ represents the phosphonium cation as shown in any of structures VI-LVI and A²⁻ represents a divalent weakly coordinating anion, are also useful.

The anion A in each of structures VI-LVI can be any weakly coordinating anion, including any of those mentioned before, in particular a monovalent anion such as tetrakis[perfluorophenyl]borate, tetrakis[3,5-bis(trifluoromethyl)phenyl]borate and trifluoromethanesulfonate (triflate) being preferred.

Specific examples of phosphorus catalysts having structure IV or V include: Cp*P⁺² (A-)₂, Cp*PF⁺¹ A-, Cp*PCl⁺¹ A-, Cp*PBr⁺¹ A-, Cp*POMe⁺¹ A-, Cp*POEt⁺¹ A-, Cp*PPh⁺¹ A-, wherein Cp* is as before, especially unsubstituted cyclopentadienyl, pentaflurocyclopentadienyl, pentachlorocyclopentadienyl, pentamethylcyclopentadienyl, pentaphenylcyclopentadienyl, indenyl or fluorenyl, OMe represents methoxyl, OEt represents ethoxyl, OPh represents phenoxyl and A- in each case is monovalent. Analogous compounds in which A represents a divalent weakly coordinating anion are also useful. A is most preferably a monovalent anion such as tetrakis[perfluorophenyl]borate, tetrakis[3,5-bis(trifluoromethyl)phenyl]borate and trifluoromethanesulfonate (triflate). Especially preferred phosphorus catalysts of Structure IV and V are the tetrakis[perfluorophenyl]borate salts of Cp*P⁺², Cp*PF⁺¹ and Cp*PCl⁺¹, where CP* is unsubstituted cyclopentadienyl, pentaflurocyclopentadienyl, pentachlorocyclopentadienyl, pentamethylcyclopentadienyl, pentaphenylcyclopentadienyl, indenyl or fluorenyl.

Methods useful for preparing the various phosphorus catalysts are generally described, for example, in Angew. Chem. Int. Ed. 2014, 53, 6538-6541, Chem. Sci. 2015, 6, 2016 Chem. Commun., 2018, 54, 662-665, Chem. 2018, 2699-2708. In a particular method, phosphorus catalysts of Structure I can be synthesized in several steps starting with the corresponding phosphine having the structure

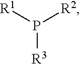

wherein R¹, R², and R³ are as defined before. Reaction with a halogenating agent yields a phosphine dihalide having the structure:

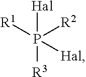

wherein Hal is F, Cl, Br or I. Examples of halogenating agents include XeF₂, perchloroethane, sulfuryl chloride, elemental bromine and elemental iodine. This reaction is conveniently performed at room temperature, or at a moderately elevated temperature (such as 50 to 100° C.), using a stoichiometric amount or small excess of the halogenating agent.

The phosphine dihalide can be converted to the corresponding phosphorium salt

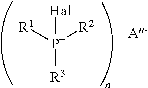

by reaction with a silylium compound having the general structure

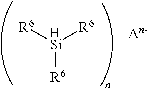

wherein each R⁶ is independently hydrocarbyl (including linear, branched and/or cyclic alkyl, aryl, aryl-substituted alkyl and alkyl-substituted aryl) and A is as defined before. The silylium compound is conveniently formed, for example, by reaction of the corresponding silane

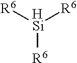

with a salt of the Aⁿ⁻ anion, such as the trityl (C⁺(C₆H₅)₃) salt. This reaction is conveniently performed in solution in a suitable solvent such as toluene at a temperature of 0 to 50° C. The product can be recovered by addition of an antisolvent (such as pentane or other liquid alkane) and if desired purified by methods such as recrystallization.

To produce a corresponding alkoxide (i.e., X in structure I is alkoxyl or inertly substituted alkoxyl), the phosphonium salt

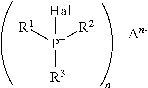

can be reacted with an anhydrous unsubstituted or inertly substituted C₁₋₁₂ alcohol.

In cases in which X in Structure I is CF₃, a suitable synthetic route starts with

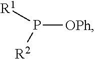

where R¹ and R² are as described above, and Ph denotes phenyl. Reaction with trimethylsilane-CF₃ in the presence of CsF replaces the phenoxy group with CF₃. Subsequent reaction with R³OTf (where OTf denotes triflate and R³ is as described before) in the presence of a palladium catalyst produces

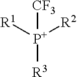

in the triflate salt form.

Double metal cyanide catalyst complexes are well known and described, for example, U.S. Pat. Nos. 3,278,457, 3,278,458, 3,278,459, 3,404,109, 3,427,256, 3,427,334, 3,427,335, and 5,470,813, among many others. The double metal cyanide catalyst complexes include an M¹ metal hexacyanometallate component, wherein the M¹ metal is, for example, Zn²⁺, Fe²⁺, Co²⁺, Ni²⁺, Mo⁴⁺, Mo⁶, Al³, V⁴⁺, V⁵⁺, Sr²⁺, W⁴⁺, W⁶⁺, Mn²⁺, Sn²⁺, Sn⁴⁺, Pb²⁺, Cu²⁺, La³⁺ or Cr³⁺, especially Zn²⁺ or Fe²⁺. The hexacyanometallate contains one or more of Fe³⁺, Fe²⁺, Co³⁺, Co²⁺, Cr²⁺, Cr³⁺, Mn²⁺, Mn³⁺, Ir³⁺, Ni²⁺, Rh³⁺, Ru²⁺, V⁴⁺, V⁵⁺, Pd²⁺, and Pt²⁺, especially Fe³⁺, Fe²⁺, Co³⁺, Co²⁺. Zinc hexacyanometallate complexes are greatly preferred. The catalyst complex may contain additional components such as additional M¹ metal compounds, low molecular weight alcohols such as t-butanol, polyethers, and the like. The catalyst complex may contain and/or be used in combination with a promoter compound, as described for example, in U.S. Pat. No. 9,040,657, WO2018/209069 and WO 2020/131508. Preferred promoter compounds include compounds of aluminum, gallium, hafnium, iron and indium, particularly oxides, hydrides and alkoxides thereof.

A commercially available double metal cyanide catalyst complex is available from Covestro LLC, Pittsburgh, Pennsylvania US as the Arcol® 3 Catalyst.

In some embodiments, the double metal cyanide catalyst corresponds to the formula:

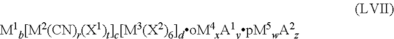

wherein:
M¹ and M⁴ each represent a metal ion independently selected from Zn²⁺, Fe²⁺, Co⁺²⁺, Ni²⁺, Mo⁴⁺, Mo⁶⁺, Al⁺³⁺, V⁴⁺, V⁵⁺, Sr²⁺, W⁴⁺, W⁶⁺, Mn²⁺, Sn²⁺, Sn⁴⁺, Pb²⁺, Cu²⁺, La³⁺, and Cr³⁺; M² and M³ each represent a metal ion independently selected from Fe³⁺, Fe²⁺, Co³⁺, Co²⁺, Cr²⁺, Cr³⁺, Mn²⁺, Mn³⁺, Ir³⁺, Ni²⁺, Rh³⁺, Ru²⁺, V⁴⁺, V⁵⁺, Ni²⁺, Pd²⁺, and Pt²⁺;
M⁵ represents one or more of gallium, hafnium, manganese, titanium, indium, aluminum, magnesium, manganese, scandium, molybdenum, cobalt, tungsten, iron, vanadium, tin, titanium, silicon and zinc and is different from M⁵;
X¹ represents a group other than cyanide that coordinates with the M² ion;
X² represents a group other than cyanide that coordinates with the M³ ion;
A¹ represents a halide, nitrate, sulfate, carbonate, cyanide, oxalate, thiocyanate, isocyanate, perchlorate, isothiocyanate, an alkanesulfonate, an arylenesulfonate, trifluoromethanesulfonate, or a C₁₋₄ carboxylate;
A² represents least one alkoxide, aryloxy, carboxylate, acyl, pyrophosphate, phosphate, thiophosphate, amide, oxide, siloxide, hydride, carbamate, halide or hydrocarbon anion; b, c and d are each numbers that reflect an electrostatically neutral complex, provided that b and c each are greater than zero;
x and y are integers that balance the charges in the metal salt M⁴ₓA¹_{y};
r is an integer from 4 to 6;
t is an integer from 0 to 2;
o is a number from 0 and 20;
p is a number from 0.001 to 20; and
w and z are numbers that balance the charges in the metal salt M⁵_{w}A²_{z}, provided that w is from 1 to 4.

M¹ and M⁴ are preferably zinc; M² and M³ are preferably cobalt. M⁵ is preferably one or more of aluminum, gallium, hafnium, indium and iron and may be a mixture of aluminum and/or iron with one or more of gallium, hafnium and indium. Double metal cyanide catalysts corresponding to formula LVII are conveniently made by precipitating the double metal cyanide (i.e., the M¹_{b}[M²(CN)ᵣ(X¹)ₜ]_{c}[M³(X²)₆]_{d} portion of the complex) in the presence of a M⁵_{w}A²_{z} compound or precursor thereto, as described in WO 2020/131508.

M⁵_{w}A²_{z} compounds in which M⁵ is aluminum include aluminum oxide, trialkyl aluminum compounds such as trimethylaluminum, triethyl aluminum, tributyl aluminum, tribenzylaluminum and the like; aluminum alkoxides such as aluminum trimethoxide, aluminum triethoxide, aluminum triisopropoxide, aluminum tri-t-butoxide, aluminum tri-sec-butoxide and the like; aluminum aryloxides such as aluminum phenoxide and aluminum phenoxides in which one or more of the phenoxide groups is ring-substituted with one or more of alkyl, CF₃, cyano, COCH₃, halogen, hydroxyl, alkoxyl and the like; aluminum oxide; aluminum carboxylates such as aluminum formate, aluminum acetate, aluminum propionate, aluminum 2-ethylhexanoate, aluminum benzoate, aluminum benzoates in which one or more of the benzoate groups is ring-substituted with one or more of alkyl, CF₃, cyano, COCH₃, halogen, hydroxyl, alkoxyl and the like, aluminum salicylate, aluminum 3,5-di-t-butyl salicylate; aluminum amides such as aluminum tris(dimethylamide), aluminum tris(diethylamide), aluminum tris(diphenylamide), aluminum tris(di(trimethylsilyl)amide) and the like; aluminum acetylacetonate; aluminum t-butylacetylacetonate; and alkylaluminum oxides and alkoxides such as diethylaluminum ethoxide, dimethylaluminum ethoxide, diethylaluminum isopropoxide, dimethylaluminum isopropoxide, methyl aluminoxane, and tetraethyldialuminoxane.

M⁵_{w}A²_{z} compounds in which M⁵ is gallium include gallium oxide, trialkyl gallium compounds such as trimethylgallium, triethyl gallium, tributyl gallium, tribenzylgallium and the like; gallium oxide; gallium alkoxides such as gallium trimethoxide, gallium triethoxide, gallium triisopropoxide, gallium tri-t-butoxide, gallium tri-sec-butoxide and the like; gallium aryloxides such as gallium phenoxide and gallium phenoxides in which one or more of the phenoxide groups is ring-substituted with one or more of alkyl, CF₃, cyano, COCH₃, halogen, hydroxyl, alkoxyl and the like; gallium carboxylates such as gallium formate, gallium acetate, gallium propionate, gallium 2-ethylhexanoate, gallium benzoate, gallium benzoates in which one or more of the benzoate groups is ring-substituted with one or more of alkyl, CF₃, cyano, COCH₃, halogen, hydroxyl, alkoxyl and the like, gallium salicylate, gallium 3,5-di-t-butyl salicylate; gallium amides such as gallium tris(dimethylamide), gallium tris(diethylamide), gallium tris(diphenylamide), gallium tris(di(trimethylsilyl)amide) and the like; gallium acetylacetonate; gallium t-butylacetylacetonate; and alkylgallium alkoxides such as diethylgallium ethoxide, dimethylgallium ethoxide, diethylgallium isopropoxide and dimethylgallium isopropoxide.

M⁵_{w}A²_{z} compounds in which M⁵ is hafnium include hafnium oxide, hafnium alkyls such as such as tetraethyl hafnium, tetrabutyl hafnium, tetrabenzyl hafnium and the like; hafnium oxide; hafnium alkoxides such as hafnium tetramethoxide, hafnium tetraethoxide, hafnium tetraisopropoxide, hafnium tetra-t-butoxide, hafnium tetra-sec-butoxide and the like; hafnium aryloxides such as hafnium phenoxide and hafnium phenoxides in which one or more of the phenoxide groups is ring-substituted with one or more of alkyl, CF₃, cyano, COCH₃, halogen, hydroxyl, alkoxyl and the like; hafnium carboxylates such as hafnium formate, hafnium acetate, hafnium propionate, hafnium 2-ethylhexanoate, hafnium benzoate, hafnium benzoates in which one or more of the benzoate groups is ring-substituted with one or more of alkyl, CF₃, cyano, COCH₃, halogen, hydroxyl, alkoxyl and the like, hafnium salicylate, hafnium 3,5-di-t-butyl salicylate; hafnium amides such as hafnium tetra(dimethylamide), hafnium tetra(diethylamide), hafnium tetra(diphenylamide), hafnium tetra((bistrimethylsilyl)amide); hafnium acetylacetonate and hafnium t-butylacetylacetonate.

M⁵_{w}A²_{z} compounds in which M⁵ is indium include indium oxide, trialkyl indium compounds like trimethyl indium; indium oxide; indium alkoxides such as indium methoxide, indium ethoxide, indium isopropoxide, indium t-butoxide, indium sec-butoxide and the like; indium aryloxides such as indium phenoxide and indium phenoxides in which one or more of the phenoxide groups is ring-substituted with one or more of alkyl, CF₃, cyano, COCH₃, halogen, hydroxyl, alkoxyl and the like; indium carboxylates such as indium formate, indium acetate, indium propionate, indium 2-ethylhexanoate, indium benzoate, indium benzoates in which one or more of the benzoate groups is ring-substituted with one or more of alkyl, CF₃, cyano, COCH₃, halogen, hydroxyl, alkoxyl and the like, indium salicylate, indium 3,5-di-t-butyl salicylate; indium acetylacetonate; and indium t-butylacetylacetonate.

In other embodiments, step (III) of the process of the invention is performed in the presence of a double metal cyanide and a separate promoter compound corresponding to M⁵_{w}A²_{z}, as described in U.S. Pat. No. 9,040,657. In such embodiments, the promoter compound is not present during the precipitation of the double metal cyanide catalyst, but instead is a separate material from the double metal cyanide catalyst. In such embodiments, the double metal cyanide is most preferably a zinc cyanometallate. The promoter compound (M⁵_{w}A²_{z}) is as described immediately above, wherein M⁵ is preferably one or more of aluminum, gallium, hafnium, indium and iron, with specific examples of M⁵_{w}A²_{z} compounds being as described above with regard to formula LVII.

In this invention, a polyether is produced in an alkoxylation process that includes at least two steps. In the first step, one or more starter compounds is alkoxylated in the presence of one or more phosphorus catalysts as described above. This produces an intermediate alkoxylated product having a number average molecular weight of 400 to 1500 g/mol, especially 400 to 1000 g/mol or 400 to 800 g/mol. The intermediate alkoxylated product contains phosphorus catalyst residues. The intermediate alkoxylated product is then further alkoxylated in the presence of the phosphorus catalyst residues and the double metal cyanide catalyst complex to produce a polyether having a number average molecular weight at least 200 g/mol greater than that of the intermediate alkoxylated product.

The starter compound has a molecular weight of up to 300 g/mol, preferably up to 250 g/mol or up to 200 g/mol and one or more functional groups capable of being alkoxylated. The invention has particular advantages when the starter compound is a low molecular weight alcohol or polyol having a hydroxyl equivalent weight of up to 75 or up to 50 g/equivalent) and for that reason prior to alkoxylation has a high concentration of hydroxyl groups. The starter in some embodiments contains 1 to 8, 1 to 6, 2 to 6, 2 to 4 or 2 to 3 functional groups per molecule. The functional groups may be, for example, primary, secondary or tertiary hydroxyl, or thiol. In certain embodiments, the functional groups are all hydroxyl groups.

Equivalent weight of starters and other alcohols or polyols is conveniently determined using titration methods such as ASTM 4274-21, which yield a hydroxyl number in mg KOH/gram of polyol that can be converted to equivalent weight using the relation equivalent weight=56,100÷hydroxyl number. Molecular weights of starters are formula molecular weights for defined compounds and number average molecular weights as measured by gel permeation chromatography against polystyrene standards for polymeric starters.

Among the suitable starters are vinyl alcohol, propenyl alcohol, allyl alcohol, acrylic acid, hydroxyethyl acrylate, hydroxyethyl methacrylate, a C₁₋₅₀ alkanol, especially a C₁₋₁₂ alkanol, phenol, cyclohexanol, an alkylphenol, water (considered for purposes of this invention as having two hydroxyl groups), ethylene glycol, diethylene glycol, triethylene glycol, propylene glycol, dipropylene glycol, tripropylene glycol, 1,4-butane diol, 1,6-hexane diol, 1,8-octane diol, cyclohexane dimethanol, glycerin, trimethylolpropane, trimethylolethane, pentaerythritol, sorbitol, sucrose, xylitol, mannitol, maltitol, sucralose, phenol, polyphenolic starters such as bisphenol-A or 1,1,1-tris(hydroxyphenyl)ethane, and the like. Another advantage of the invention is that starters which have melting temperatures in the range of 50° C. to 200° C., especially 130 to 200° C., are easily alkoxylated in step II of the process. Any two or more of the foregoing starters may be used together if desired.

The cyclic oxide is characterized in having a least one 3-, 4- or 5-member ring structure that contains an oxygen atom in the ring structure. Especially preferred cyclic oxides are oxiranes that have a three-member, oxygen containing ring. The cyclic oxide(s) may be, for example, ethylene oxide, 1,2-propylene oxide (generally referred to herein as “propylene oxide”), oxetane, 1,2-butene oxide, 2-methyl-1,2-butene oxide, 2,3-butene oxide, tetrahydrofuran, epichlorohydrin, hexene oxide, octene oxide, styrene oxide, divinylbenzene dioxide, a glycidyl ether such as bisphenol-A diglycidyl ether, epichlorohydrin or other polymerizable oxirane. In some embodiments, the alkylene oxide is 1,2-propylene oxide, ethylene oxide, or a mixture thereof, including, for example, a mixture of at least 50% (preferably at least 80%) by weight propylene oxide and correspondingly up to 50% (preferably up to 20%) by weight ethylene oxide. In some embodiments, two or more alkylene oxides are polymerized simultaneously (to form random copolymers), and or the composition of the alkylene oxide is changed one or more times, or even continuously, throughout the course of the polymerization to form block and/or random/block copolymers.

A first part of the alkoxylation is performed by combining the starter and phosphorus catalyst with the cyclic oxide(s) (Step I) and optionally comonomer and (Step II), reacting the cyclic oxide(s) with the starter by subjecting the resulting reaction mixture to reaction conditions. The catalyst may be added as a solution in a solvent or a suspension in a diluent if desired. Such a solvent preferably is inert under the conditions of the alkoxylation reaction. Diethyl ether, dichloromethane and hydrocarbons such as toluene or hexane are useful solvents or suspension for the phosphorus catalyst.

The first part of the alkoxylation proceeds at a wide range of temperatures from −100° C. to 250° C. or more. In some embodiments, the reaction temperature is at least 80° C., at least 100° C., at least 120° C., at least 130° C. or at least 150° C. The temperature in (Step II) preferably does not exceed 190° C., and more preferably does not exceed 180° C. An important advantage of the phosphorus catalysts is they perform well without premature deactivation at higher temperatures, especially 150° to 200° C. or 150° to 180° C. The higher temperatures promote faster reactions. Additionally, the ability to operate at these higher temperatures permits the process to be used with starters and/or cyclic oxides that have somewhat high melting temperatures (such as sorbitol, xylitol, mannitol, maltitol, sucralose) and/or which are viscous at lower temperatures, or which, like sorbitol and glycerin, have limited solubility in the cyclic oxide at lower temperatures.

The alkoxylation reaction usually is performed at a superatmospheric pressure but can be performed at atmospheric pressure or even a subatmospheric pressure.

Enough phosphorus catalyst is used to provide a commercially reasonable alkoxylation rate, but it is generally desirable to use as little thereof as possible consistent with reasonable alkoxylation rates, as this both reduces the cost for the catalyst and can eliminate the need to remove catalyst residues from the product. The amount of phosphorus catalyst may be, for example, sufficient to provide 10 to 10,000 ppm by weight of phosphorus catalyst based on the weight of the starter. In specific embodiments, the amount of phosphorus catalyst may be sufficient to provide at least 25 ppm, at least 50 ppm or at least 100 ppm catalyst on the foregoing basis, and up to 1,000 ppm or up to 500 ppm catalyst, again on the foregoing basis. The weight of the phosphorus catalyst includes the weight of both cation and associated anion.

Alkoxylation (Step II) in the presence of the phosphorus catalyst is continued until an intermediate alkoxylated product having a number average molecular weight of 400 to 1500, especially 400 to 1000 or 400 to 800 g/mol.

In (Step III) of the process, the intermediate alkoxylated product together with the phosphorus catalyst residues is further alkoxylated by reaction with cyclic oxide in the presence of a catalytically effective amount of a double metal cyanide catalyst complex. The ability to further alkoxylate in the presence of the phosphorus catalyst residues is an important and unexpected advantage of the invention. The intermediate alkoxylated product may be taken from Step II into Step III of the process without performing a removal step to remove the phosphorus catalyst residues prior to performing Step III. If desired, unreacted cyclic oxide(s) from Step II of the process may be used as some or all of the cyclic oxide in Step III of the process.

In Step III a polyether is produced having a number average molecular weight at least 200 g/mol greater than that of the intermediate alkoxylated product. The polyether molecular weight may be, for example, 800 to 12,000 g/mol, 1000 to 8,000 g/mol, 1000 to 6000 g/mol or 1000 to 5000 g/mol, for example.

The amount of double metal cyanide catalyst used in Step III may be, for example 5 to 1000, especially 5 to 250 ppm by weight based on the weight of the product polyether.

Reaction conditions in Step III are generally as described with regard to Step II, with a reaction temperature of 80 to 180° C. being preferred.

An especially preferred cyclic oxide in Step III is 1,2-propylene oxide by itself. With this invention, the proportion of primary hydroxyl groups formed when 1,2-propylene oxide is homopolymerized in step (III) is often unexpectedly high, often being 15 to 25% or even more.

Either or both of Steps II and II can be performed batch-wise, semi-continuously (including with continuous addition of starter as described in U.S. Pat. No. 5,777,177) or continuously.

The alkoxylation reactions can be performed in any type of vessel that is suitable for the pressures and temperatures encountered. The reactor should be equipped with a means of providing and/or removing heat, so the temperature of the reaction mixture can be maintained within the required range. Suitable means include various types of jacketing for thermal fluids, various types of internal or external heaters, and the like. A cook-down step performed on continuously withdrawn product is conveniently conducted in a reactor that prevents significant back-mixing from occurring. Plug flow operation in a pipe or tubular reactor is a preferred manner of performing such a cook-down step.

The crude polyether obtained in any of the foregoing processes may contain unreacted cyclic oxide, small quantities of the starter compound and low molecular weight alkoxylates thereof, and small quantities of other organic impurities and/or water. Volatile impurities (including unreacted cyclic oxides) should be flashed or stripped from the product. The crude product typically contains residues of both the phosphorus catalyst and the double metal cyanide catalyst complex. It is typical to leave these residues in the product, but these can be removed if desired. Moisture and volatiles can be removed by stripping the alkoxylated product. Stripping can be performed under conditions of elevated temperature, subatmospheric pressure, and/or dynamic agitation, as desired.

The process of the invention is useful for preparing alkoxylated products that can have hydroxyl equivalent weights of 800 to 12,000 g/mol, 1000 to 8,000 g/mol, 1000 to 6000 g/mol or 1000 to 5000 g/mol. Alkoxylated polyols produced in accordance with the invention are useful raw materials for producing polyurethanes and other polymers made by reacting the alkoxylated polyol with a polyisocyanate. These products include a wide variety of cellular and non-cellular materials, which may vary in physical properties from very rigid to highly flexible. Alkoxylated monols produced in accordance with the invention are useful as surfactants or as industrial solvents, among other uses. Alkoxylated polyols and monols can be aminated to produce the corresponding amine-terminated materials, which are in turn useful raw materials for making various materials including polyureas and cured epoxy resins.

In some embodiments the cyclic oxide is polymerized in Step II, Step III or both with or in the presence of one or more copolymerizable monomers that are not cyclic oxides. Examples of such copolymerizable monomers include carbonate precursors that copolymerize with an alkylene oxide to produce carbonate linkages in the product. Examples of such carbonate precursors include carbon dioxide, phosgene, linear carbonates and cyclic carbonates. Other copolymerizable monomers include carboxylic acid anhydrides, which copolymerize with cyclic oxides to produce ester linkages in the product.

The following examples are provided to illustrate the invention but are not intended to limit the scope thereof. All parts and percentages are by weight unless otherwise indicated.

### Catalyst Preparation Procedure

The tetrakis(pentafluorophenyl) borate salt of phosphonium dicatecholate,

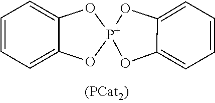 [SMILES: c1ccc2c(c1)O[P+]1(O2)Oc2ccccc2O1]

is made according to the method described in J. Am. Chem. Soc. 2021, 143, 15845-15851.

P(PFP)₃F tetrakis(pentafluorophenyl)borate is made by reacting tris(perfluorophyl) phosphine (P(PFP)₃) with XeF₂ in the general manner described in Science 341, 1374 (2013) to produce P(PFP)₃F₂. The product is recovered and recrystallized, and its structure confirmed by ¹H, ¹³C and ³¹P NMR. The P(PFP)₃F₂ is suspended in toluene at room temperature. Separately, a silylium solution is produced by combining triethyl silane and trityl tetrakis(pentafluorophenyl)borate in toluene. The P(PFP)₃F₂ suspensions and silylium solutions are combined at room temperature and stirred for 30 minutes. The toluene is removed by evaporation to produce a slurry, which is triturated with pentane until it solidified. The product is then recrystallized from dichloromethane using pentane as an antisolvent.

P(2-F)₃F tetrakis(pentafluorophenyl)borate,

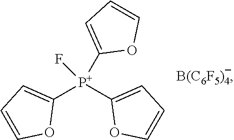

is made by reacting tris(2-furyl) phosphine with XeF₂ in the general manner described in Chem. Sci. 2015, 6, 2016 to produce P(2-F)₃F₂. The P(2-F)₃F₂ is suspended in toluene at room temperature. Separately, a silylium solution is produced by combining triethyl silane and trityl tetrakis(pentafluorophenyl)borate in toluene. The P(2-F)₃F₂ suspension and silylium solution are combined at room temperature and stirred for 30 minutes. The toluene is removed by evaporation to produce a slurry, which is triturated with pentane until it solidified. The product is then recrystallized from dichloromethane using pentane as an antisolvent. The product is recovered and recrystallized, and its structure confirmed by ¹H, ¹³C and ³¹P NMR.

The tetrakis(pentafluorophenyl) borate salt of PMCp*P²⁺, where PMCp* is pentamethylcyclopentadienyl, is prepared in the general manner described in Chem. 4, 2699-2708. The PMCp*P²⁺ dication may have one or more toluene ligands coordinated to the structure.

Double Metal Cyanide Complex 1 is made in the general manner described in Example 1 of WO2018/209069. The catalyst complex contains 24% by weight zinc, 10.5% cobalt, 1.6% aluminum, a zinc:cobalt molar ratio of 2.09 and an aluminum:cobalt molar ratio of 0.35, all as determined by neutron activation analysis.

Double Metal Cyanide Complex 2 is a zinc hexacyanocobaltate catalyst complex sold commercially as ARCOL® 3 catalyst.

### EXAMPLES 1-4 AND COMPARATIVE SAMPLES A-B

Example 1: Molten sorbitol is alkoxylated by combining it with 350 ppm (initial concentration) of catalyst (PCat₂) tetrakis(pentafluorophenyl)borate and 154.7 mL propylene oxide and heating under nitrogen at 160° C. for 1.2 hours. This produces a propoxylated sorbitol intermediate having a number average molecular weight of 575 g/mol, by gel permeation chromatography against polystyrene standards.

76.4 g of the resulting intermediate is transferred to an autoclave reactor without removal of phosphorus catalyst residues. 60 mg of the Double Metal Cyanide Complex 1 are added. After nitrogen sparging, the reactor and its contents are heated to 130° C. and 324 propylene oxide fed over 3.6 hours to produce a 3000 number average molecular weight polyether. 17.7% of the hydroxyl groups of the product polyether are primary.

Examples 2-4 are made in the same general manner except the phosphorus catalyst in Example 2 is P(PFP)₃F tetrakis (pentafluorophenyl)borate, the phosphorus catalyst in Example 3 is P(2-F)₃F tetrakis(pentafluorophenyl)borate, and the phosphorus catalyst in Example 4 is the tetrakis(pentafluorophenyl) borate salt of PMCp*P²⁺, where PMCp* is pentamethylcyclopentadienyl. In each case, a 3000 molecular weight product is obtained by adding about 325 g of propylene oxide in the second polymerization step. 16.3% of the hydroxyl groups of the polyethers produced in Examples 2 and 3 are primary. Primary hydroxyl content of the product of Example 4 is not determined.

Example 5 is made in the same general manner as Example 1 except Double Metal Cyanide Complex 2 replaces Double Metal Cyanide Complex 1. Once again, a 3000 molecular weight product is obtained by adding about 325 g of propylene oxide in the second polymerization step.

Comparative Sample A is made in the same manner as Example 1, except the double metal cyanide catalyst is not added into the second reaction step. Instead, the polymerization is continued in the presence of the phosphorus catalyst. Only 159.8 mL of propylene oxide are fed and reacted during the second polymerization step, resulting in a polyether product having a molecular weight far below the target.

Comparative Sample B is made by loading 337.16 g of a 700 molecular weight propoxylated sorbitol and 3.4 g of sorbitol into the autoclave reactor. 52.5 mg of the double metal cyanide catalyst complex is added. After nitrogen sparging, the reactor and its contents are heated to 160° C., and 9.4 g of propylene oxide are added. No apparent polymerization occurs by this time, so the run is discontinued.

1. An alkoxylation process, comprising the following steps:
(I) forming a reaction mixture comprising; a) a starter compound having at least one hydroxyl or thiol group and a molecular weight of up to 300, b) at least one cyclic oxide, and c) a catalytically effective amount of a phosphorus catalyst;
(II) reacting the cyclic oxide b) with the starter compound a) in the presence of the phosphorus catalyst c) to form an intermediate alkoxylated product having a number average molecular weight of 400 to 1500 and containing residues of the phosphorus catalyst; and
(III) further alkoxylating the intermediate alkoxylated product containing residues of the phosphorus catalyst in the presence of a catalytically effective amount of a double metal cyanide catalyst complex to produce a polyether having a number average molecular weight at least 200 g/mol greater than that of the intermediate alkoxylated product;
wherein the phosphorus catalyst c) has any of the following structures; 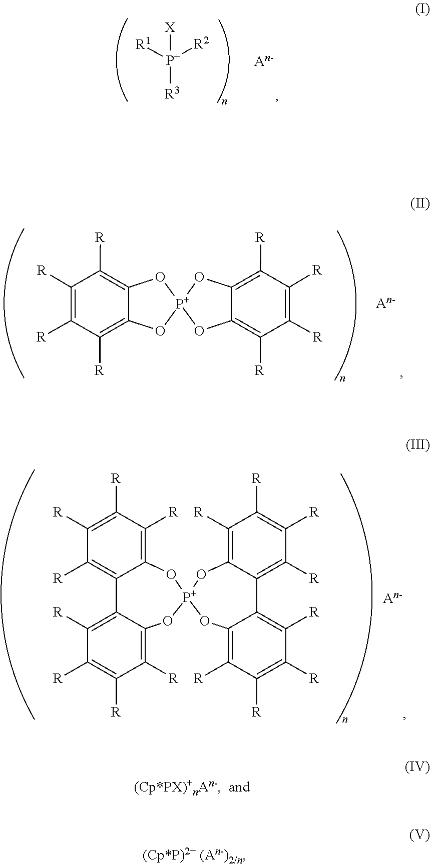
wherein R¹, R² and R³ independently are groups having unsubstituted or substituted, optionally heteroatomic, aromatic five- or six-member rings having a direct bond between a carbon atom of the optionally heteroatomic aromatic five- or six-member ring and the phosphorus atom, each R is independently hydrogen, halogen, unsubstituted or inertly substituted C₁₋₁₂ alkoxyl, aryloxy, unsubstituted or inertly substituted linear, branched and/or cyclic alkyl and unsubstituted or inertly substituted aryl, with the proviso that any two R groups may together form a ring structure, Cp* designates an optionally substituted cyclopentadienyl ligand, X is halogen, hydroxyl, unsubstituted or inertly substituted alkyl, unsubstituted or inertly substituted aryl, unsubstituted or inertly substituted alkoxy, or unsubstituted or inertly substituted aryloxy, A represents a weakly coordinating anion, n− represents the valence of A, and n represents the absolute value of the valence of A.

2. The alkoxylation process of claim 1, wherein the phosphorus catalyst c) comprises one or more of: 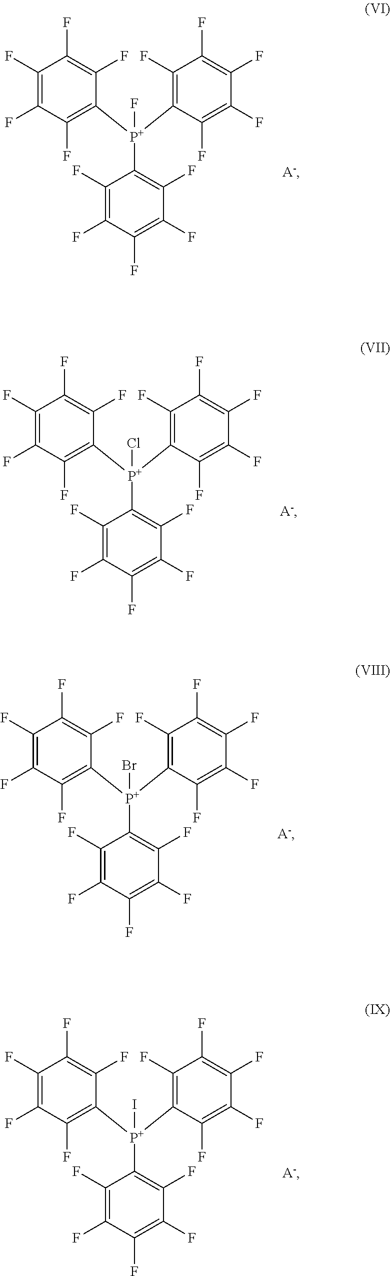 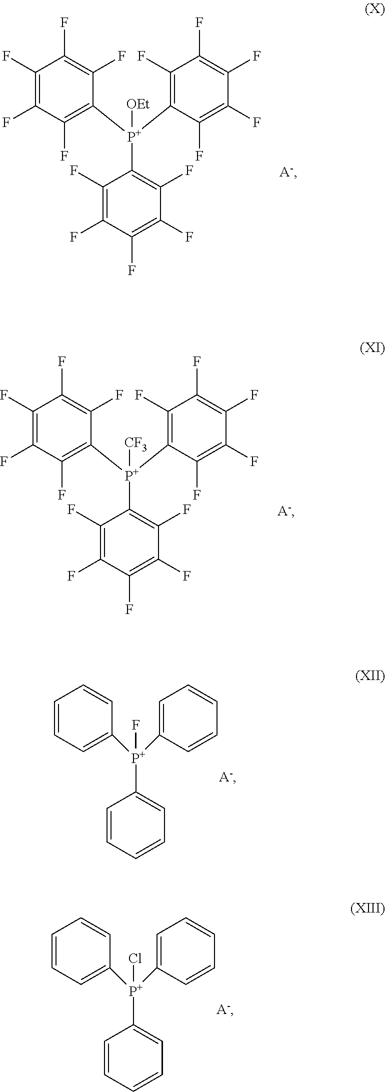 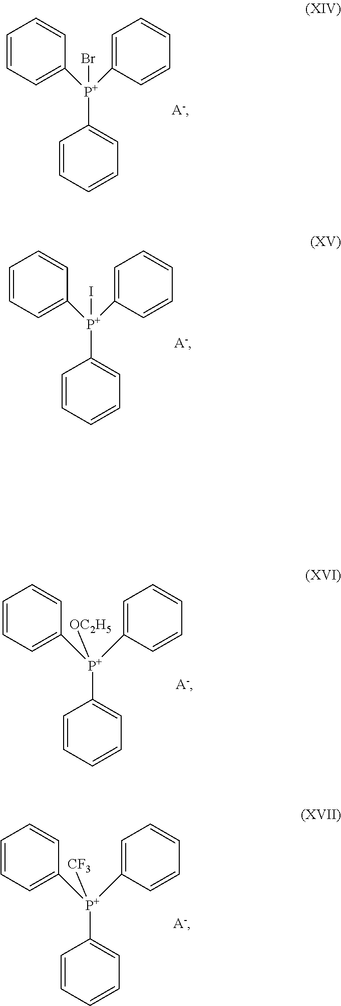 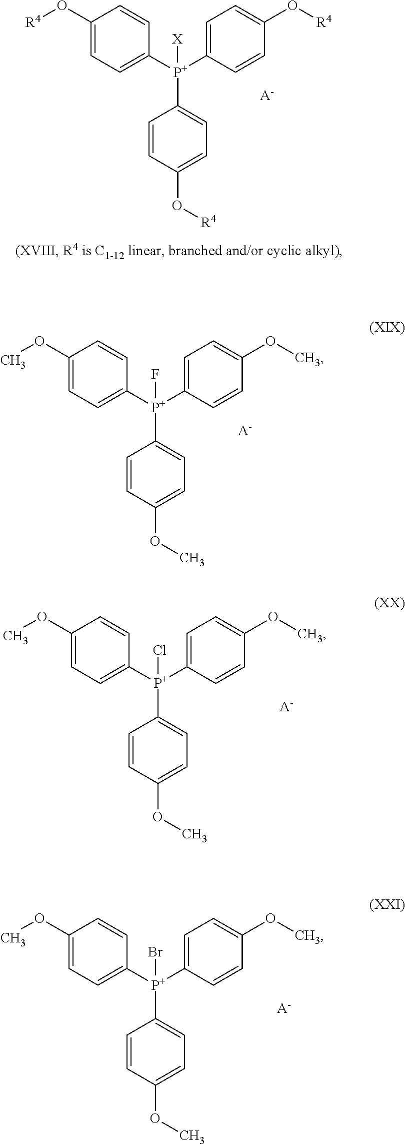 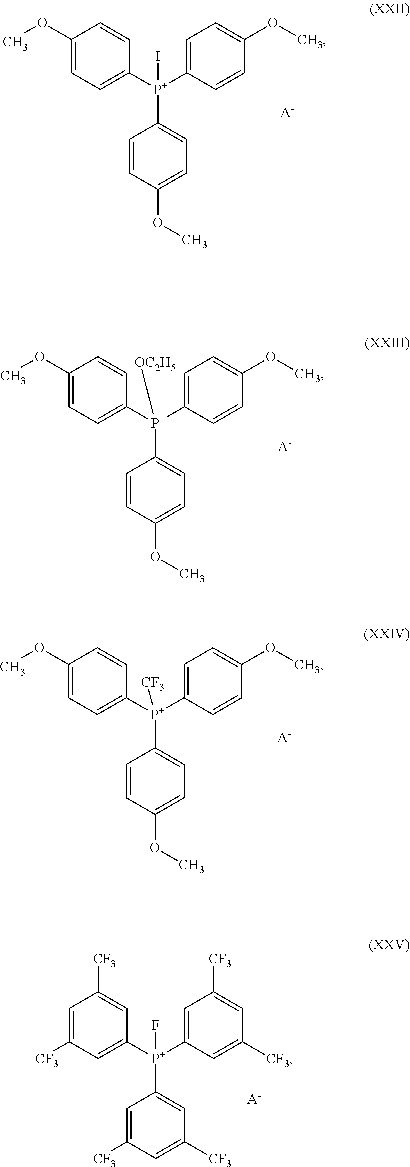 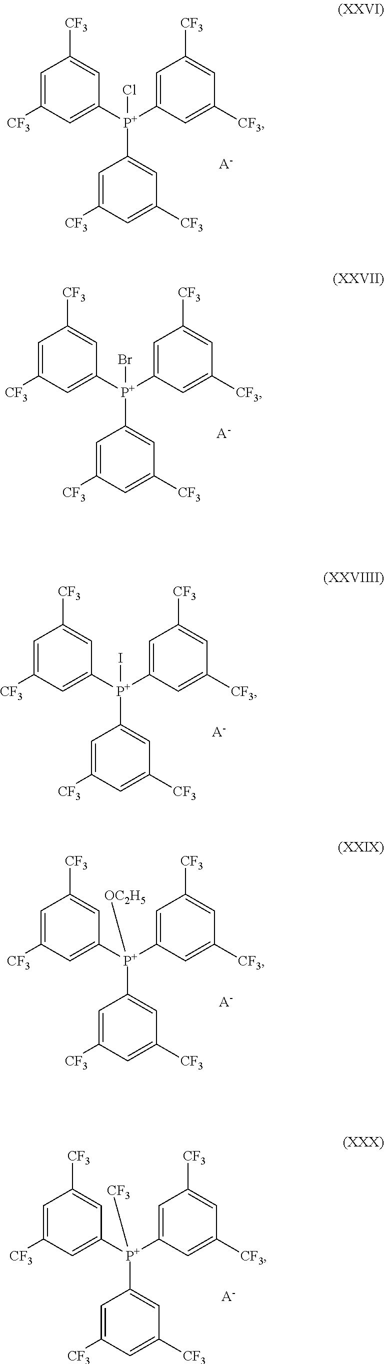 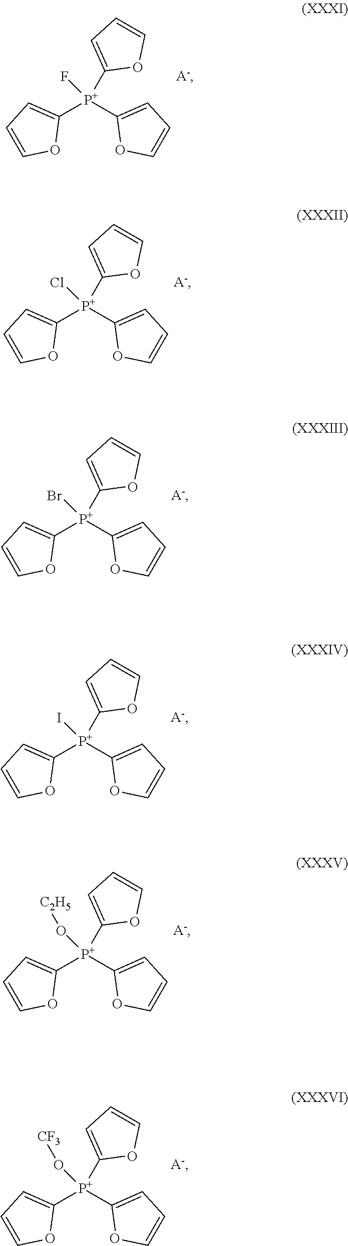 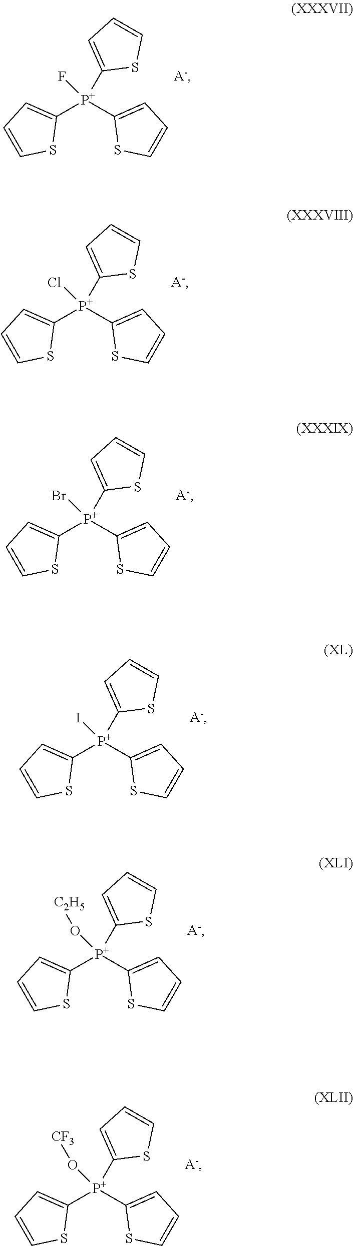 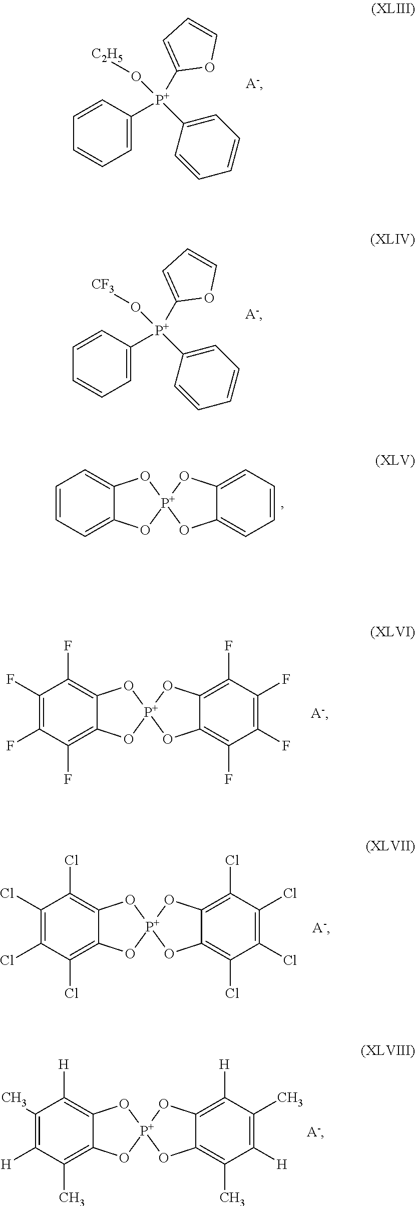 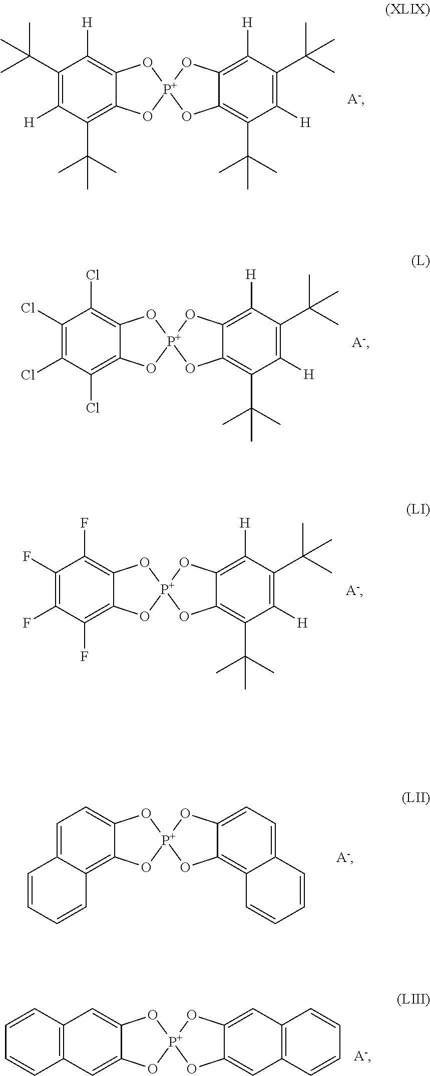 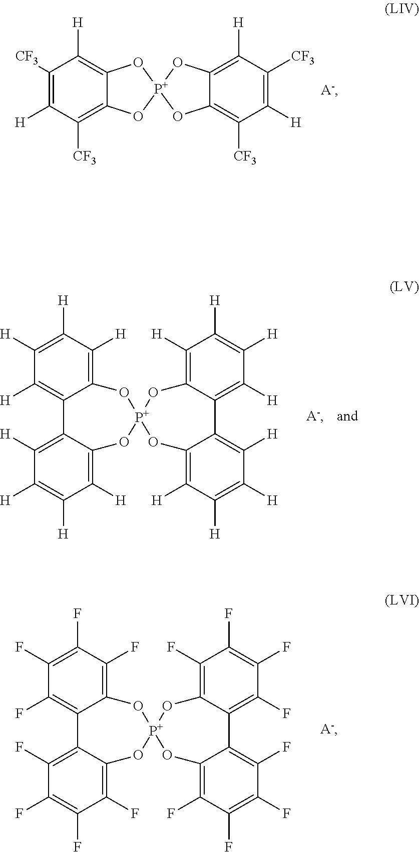
wherein for each case, A⁻ is a monovalent anion.

3. The alkoxylation process of claim 1, wherein the phosphorus catalyst (c) comprises one or more of Cp*P⁺²(A⁻)₂, Cp*PF⁺¹ A⁻, Cp*PCl⁺¹A⁻, Cp*PBr⁺¹A⁻, Cp*POMe⁺¹A⁻, Cp*POEt⁺¹A⁻, and Cp*PPh⁺¹A⁻, wherein Cp* is unsubstituted cyclopentadienyl, pentaflurocyclopentadienyl, pentachlorocyclopentadienyl, pentamethylcyclopentadienyl, pentaphenylcyclopentadienyl, indenyl or fluorenyl, OMe represents methoxy, OEt represents ethoxy, OPh represents phenoxyl, and A represents a weakly coordinating anion.

4. The alkoxylation process of claim 3, wherein the phosphorus catalyst (c) comprises a tetrakis[perfluorophenyl]borate salt of Cp*P⁺², Cp*PF⁺¹ or Cp*PCl⁺¹, where CP* is unsubstituted cyclopentadienyl, pentaflurocyclopentadienyl, pentachlorocyclopentadienyl, pentamethylcyclopentadienyl, pentaphenylcyclopentadienyl, indenyl or fluorenyl.

5. The alkoxylation process of claim 1, wherein the double metal cyanide catalyst complex corresponds to the formula: 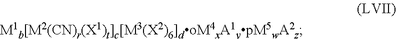
wherein:
M¹ and M⁴ each represent a metal ion independently selected from Zn²⁺, Fe²⁺, Co⁺²⁺, Ni²⁺, Mo⁴⁺, Mo⁶⁺, Al⁺³⁺, V⁴⁺, V⁵⁺, Sr²⁺, W⁴⁺, W⁶⁺, Mn²⁺, Sn²⁺, Sn⁴⁺, Pb²⁺, Cu²⁺, La³⁺, and Cr³⁺;
M² and M³ each represent a metal ion independently selected from Fe³⁺, Fe²⁺, Co³⁺, Co²⁺, Cr²⁺, Cr³⁺, Mn²⁺, Mn³⁺, Ir³⁺, Ni²⁺, Rh³⁺, Ru²⁺, V⁴⁺, V⁵⁺, Ni²⁺, Pd²⁺, and Pt²⁺;
M⁵ represents one or more of gallium, hafnium, manganese, titanium, indium, aluminum, magnesium, manganese, scandium, molybdenum, cobalt, tungsten, iron, vanadium, tin, titanium, silicon and zinc and is different from M⁵;
X¹ represents a group other than cyanide that coordinates with the M² ion;
X² represents a group other than cyanide that coordinates with the M³ ion;
A¹ represents a halide, nitrate, sulfate, carbonate, cyanide, oxalate, thiocyanate, isocyanate, perchlorate, isothiocyanate, an alkanesulfonate, an arylenesulfonate, trifluoromethanesulfonate, or a C₁₋₄ carboxylate;
A² represents least one alkoxide, aryloxy, carboxylate, acyl, pyrophosphate, phosphate, thiophosphate, amide, oxide, siloxide, hydride, carbamate, halide or hydrocarbon anion;
b, c and d are each numbers that reflect an electrostatically neutral complex, provided that b and c each are greater than zero;
x and y are integers that balance the charges in the metal salt M⁴ₓA¹_{y};
r is an integer from 4 to 6;
t is an integer from 0 to 2;
o is a number from 0 and 20;
p is a number from 0.001 to 20; and
w and z are numbers that balance the charges in the metal salt M⁵_{w}A²_{z}, provided that w is from 1 to 4.

6. The alkoxylation process of claim 1, wherein step (III) is performed in the presence of the double metal cyanide catalyst complex and a separate promoter compound having the formula M⁵_{w}A²_{z} wherein M⁵ represents one or more of gallium, hafnium, manganese, titanium, indium, aluminum, magnesium, manganese, scandium, molybdenum, cobalt, tungsten, iron, vanadium, tin, titanium, silicon and zinc and is different from M⁵; A² represents least one alkoxide, aryloxy, carboxylate, acyl, pyrophosphate, phosphate, thiophosphate, amide, oxide, siloxide, hydride, carbamate, halide or hydrocarbon anion; and w and z are numbers that balance the charges in the promoter compound M⁵_{w}A²_{z}, provided that w is from 1 to 4.

7. The alkoxylation process of claim 5, wherein M⁵ is one or more of aluminum, iron, gallium, indium, and hafnium.

8. The alkoxylation process of claim 1, wherein the starter compound a) comprises one or more of vinyl alcohol, propenyl alcohol, allyl alcohol, acrylic acid, hydroxyethyl acrylate, hydroxyethyl methacrylate, a C₁₋₅₀ alkanol, optionally a C₁₋₁₂ alkanol, phenol, cyclohexanol, an alkylphenol, water, ethylene glycol, diethylene glycol, triethylene glycol, propylene glycol, dipropylene glycol, tripropylene glycol, 1,4-butane diol, 1,6-hexane diol, 1,8-octane diol, cyclohexane dimethanol, glycerin, trimethylolpropane, trimethylolethane, pentaerythritol, sorbitol, sucrose, xylitol, mannitol, maltitol, sucralose, phenol, bisphenol-A, and 1,1,1-tris(hydroxyphenyl)ethane.

9. The alkoxylation process of claim 1, wherein the starter compound a) comprises glycerin or a mixture of glycerin and sucrose.

10. The alkoxylation process of claim 1, wherein the starter compound a) comprises sorbitol.

11. The alkoxylation process of claim 1, wherein the cyclic oxide b) comprises an oxirane.

12. The alkoxylation process of claim 1, wherein the cyclic oxide b) comprises one or more of 1,2-propylene oxide, oxetane, 1,2-butene oxide, 2-methyl-1,2-butene oxide, 2,3-butene oxide, tetrahydrofuran, epichlorohydrin, hexene oxide, octene oxide, styrene oxide, divinylbenzene dioxide, a glycidyl ether, bisphenol-A diglycidyl ether, and epichlorohydrin.

13. The alkoxylation process of claim 1, wherein the cyclic oxide b) comprises 1,2-propylene oxide, ethylene oxide, or a mixture thereof.

14. The alkoxylation process of claim 1, wherein in step (III), the intermediate alkoxylated product is alkoxylated with 1,2-propylene oxide by itself.

15. The alkoxylation process of claim 14, wherein 15 to 25% of the hydroxyl groups of the polyether are primary hydroxyl groups.
